(19) United States
(12) Patent Application Publication
(10) Pub. No.: US 2014/0377225 A1
(43) Pub. Date: Dec. 25, 2014

(54) METHODS OF IMPROVING BONE HEALTH AND MUSCLE HEALTH

(71) Applicants: Bruce Alan Watkins, West Lafayette, IN (US); Kevin Mark Hannon, West Lafayette, IN (US); Allan John Lepine, Dayton, OH (US)

(72) Inventors: Bruce Alan Watkins, West Lafayette, IN (US); Kevin Mark Hannon, West Lafayette, IN (US); Allan John Lepine, Dayton, OH (US)

(21) Appl. No.: 14/480,300

(22) Filed: Sep. 8, 2014

Related U.S. Application Data
(62) Division of application No. 11/891,120, filed on Aug. 9, 2007.
(60) Provisional application No. 60/836,611, filed on Aug. 9, 2006.

(51) Int. Cl. A61K 31/202; A23L 1/30; A23L 2/52; A23L 1/39; A61K 38/16; A23K 1/18

(52) U.S. Cl. CPC A61K 31/202; A61K 38/16; A23K 1/1846; A23L 2/52; A23L 1/39; A23L 1/3008; A23V 2002/00
USPC 424/93.4; 514/560; 514/16.7; 514/21.2; 426/2

(57) ABSTRACT

The present invention comprises a method of improving bone health and muscle health in a companion animal comprising administering a composition comprising a fatty acid component to the companion animal wherein the fatty acid component comprises a ratio of docosahexaenoic acid (DHA) to eicosapentaenoic acid (EPA) from about 3:1 to about 25:1. The present invention additionally comprises a composition comprising a fatty acid component comprising docosahexaenoic acid (DHA) and eicosapentaenoic acid (EPA) wherein the ratio of docosahexaenoic acid (DHA) to eicosapentaenoic acid (EPA) is from about 3:1 to about 25:1.

### CROSS REFERENCE TO RELATED APPLICATION

This application claims the benefit of U.S. Provisional Application No. 60/836,611, filed Aug. 9, 2006.

### FIELD OF THE INVENTION

The invention is directed to methods of improving bone health and muscle health in companion animals comprising administering composition comprising a fatty acid component comprising docosahexaenoic acid (DHA) and eicosapentaenoic acid (EPA) wherein the ratio of DHA to EPA is from about 3:1 to about 25:1.

### BACKGROUND OF THE INVENTION

Poor bone health and muscle health in companion animals severely limit an animals' quality of life and affects the overall health of the companion animal. In situations where the animal cannot put its weight onto a limb or cannot actively move around, the animal will lose both muscle mass and bone density. Maintaining the muscle mass and the bone density and preventing the loss of each will benefit the bone health and muscle health as well as the overall health of the animal by decreasing recovery time from surgery, disease, and injury.

Decreased bone mineral density, bone mineral content, and bone ash content are all indicators of poor bone health. Another threat to bone health is osteopenia. Osteopenia is the result of bone resorption exceeding the rate of bone formation. As the bone becomes more porous, the bone mineral density, bone mineral content, and bone ash content decrease and the risk of developing osteopenia increases. The animal loses bone strength as the bone becomes more porous. The loss in bone strength makes the bone more likely to fracture.

Decreased muscle wet weight, muscle fiber diameter, and muscle mass are all indicators of poor muscle health. As the wet weight, muscle fiber diameter, and muscle mass decrease, the muscle is more likely to atrophy thereby causing the animal to lose strength in that muscle. A decrease in muscle health also affects the overall health of the animal. When an animal loses its strength, it is less active and prone to weight management problems.

Muscle health also affects bone health. The biomechanical forces that act on the bone are a result of muscle strength. This is the principle governing bone modeling and remodeling. Therefore, bone growth and architecture are dynamically influenced by muscle force.

There is still a need for methods and compositions that improve and treat muscle health and bone health and prevent the loss of muscle health and bone health during the life of a companion animal.

It is therefore an object of the present invention to provide compositions and methods of improving bone health and muscle health in a companion animal comprising administering a composition comprising a fatty acid component that prevents and/or treats osteopenia, bone mineral density, bone mineral content, bone ash content, muscle fiber diameter, muscle atrophy, muscle mass, and/or muscle wet weight and promotes an improved quality of life through all the life stages of the companion animal.

### SUMMARY OF THE INVENTION

The present invention is directed to a method of improving bone health and muscle health in a companion animal comprising: administering a composition comprising a fatty acid component to a companion animal; and wherein said fatty acid component comprises a ratio of docosahexaenoic acid (DHA) to eicosapentaenoic acid (EPA) from about 3:1 to about 25:1.

The present invention is further directed to a companion animal composition comprising: a composition comprising a fatty acid component comprising docosahexaenoic acid (DHA) and eicosapentaenoic acid (EPA); and wherein said fatty acid component comprises a ratio of docosahexaenoic acid (DHA) to eicosapentaenoic acid (EPA) from about 3:1 to about 25:1.

### DETAILED DESCRIPTION OF THE INVENTION

The method of improving and treating bone health and muscle health in companion animals comprises administering a composition comprising a fatty acid component comprising DHA and EPA in a ratio of from about 3:1 to about 25:1.

These and other limitations of the composition and method of the present invention, as well as many of the optional ingredients suitable for use herein, are described in detail hereinafter.

As used herein, the term “EPA” refers to eicosapentaenoic acid (all-cis-5,8,11,14,17-icosapentaenoic acid), an omega-3 fatty acid. EPA is a carboxylic acid with a 20-carbon chain and five cis double bonds with the first double bond located at the third carbon from the methyl end. It has the molecular formula C₂₀H₃₀O₂.

As used herein, the term “DHA” refers to docosahexaenoic acid (all-cis-docosa-4,7,10,13,16,19-hexaenoic acid), an omega-3 fatty acid. DHA is a carboxylic acid with a 22-carbon chain and six cis double bonds with the first double bond located at the third carbon from the methyl end. It has the molecular formula C₂₂H₃₂O₂.

As used herein, the term “omega-3 fatty acid” refers to a polyunsaturated fatty acid wherein the first double bond exists as the third carbon-carbon bond from the methyl end of the carbon chain.

As used herein, the term “omega-6 fatty acid” refers to a polyunsaturated fatty acid wherein the first double bond exists as the sixth carbon-carbon bond from the methyl end of the carbon chain.

As used herein, the term “PUFA” refers to a class of unsaturated fatty acids that contain more than one double bond in the carbon chain.

As used herein, the term “osteopenia” refers to an imbalance between bone formation and bone resorption with the rate of resorption exceeding the rate of formation thereby negatively impacting the biological and structural integrity of the bone.

As used herein, the term “muscle atrophy” refers to a loss of muscle mass.

As used herein, the term “muscle mass” refers to a quanitification of the total amount of tissue in the body or part of the body that is muscle tissue.

As used herein, the term “muscle wet weight” refers to the weight of a muscle as it exists in the in vivo environment (i.e., hydrated).

As used herein, the term “companion animal” means an animal preferably including (for example) dogs, cats, kittens, puppies, senior dogs, senior cats, adult dogs, adult cats, horses, cows, pigs, rabbits, guinea pigs, hamsters, gerbils, ferrets, zoo mammals and the like. Dogs, cats, kittens, puppies, senior dogs, senior cats, adult dogs, and adult cats are particularly preferred.

As used herein, the term “composition” means a composition that can be ingested by a companion animal, supplements for a companion animal, pet food, dog food, cat food, treats, biscuits, raw hide, chews, fillers, gravy, sauce, beverage, supplemental water, and combinations thereof. The composition can be wet, moist, and/or dry.

The term “complete and nutritionally balanced” as used herein, unless otherwise specified, refers to a product having all known required nutrients in proper amounts and proportions based upon the recommendation of recognized authorities in the field of animal nutrition.

The composition and method of the present invention can comprise, consist of, or consist essentially of, the essential elements and limitations of the invention described herein, as well as any additional or optional ingredients, components, or limitations described herein or otherwise useful in compositions intended for companion animal consumption.

All percentages, parts and ratios as used herein are by weight of the total composition, unless otherwise specified. All such weights as they pertain to listed ingredients are based on the active level and, therefore do not include solvents or by-products that may be included in commercially available materials, unless otherwise specified.

Method

The methods of the present invention comprise improving bone health and muscle health in a companion animal comprising administering a composition comprising a fatty acid component to a companion animal and wherein said fatty acid component comprises a ratio of docosahexaenoic acid (DHA) to eicosapentaenoic acid (EPA) from about 3:1 to about 25:1. Improving bone health comprises preventing and/or treating osteopenia and/or preventing and/or treating the loss of bone mineral density and/or preventing and/or treating the loss of bone mineral content and/or preventing and/or treating the loss of bone ash content. Improving muscle health comprises preventing and/or treating muscle atrophy and/or preventing and/or treating the loss of muscle fiber diameter and/or preventing and/or treating the loss of muscle wet weight and/or preventing and/or treating the loss of muscle mass.

Composition Form

The composition of the present invention is adapted for use by companion animals. The composition of the present invention is preferably administered to improve bone health and muscle health.

The composition of the present invention can be a moist composition (i.e. those having a total moisture content of from about 16% to 50%, by weight of the product), and/or a wet composition (i.e. those having a total moisture content of greater than 50%, by weight of the product), and/or dry composition (i.e. those having a total moisture content of from about 0% to about 16%, by weight of the product). Unless otherwise described herein, wet composition, moist composition and/or dry composition are not limited by their composition or method of preparation.

The composition herein can be complete and nutritionally balanced. A complete and nutritionally balanced composition may be compounded to be fed as the sole ration and is capable of maintaining the life and/or promote reproduction without any additional substance being consumed, except for water.

The composition and components of the present invention are selected for consumption by a companion animal and are not intended for consumption by humans. Non-limiting examples of compositions include supplements for an animal, pet food, dog food, cat food, treats, biscuits, raw hide, treats, chews, fillers gravy, sauce, beverage, supplemental water, pills, tablets, capsules, and combinations thereof.

Additionally, administration in accordance with the present invention may be continuous or intermittent, depending, for example, upon the recipient's physiological condition, whether the purpose of the administration is therapeutic or prophylactic, and other factors known to skilled practitioners. The composition utilized in accordance with the present invention can additionally be formulated as a pharmaceutical and/or veterinary composition and administered to an animal in a variety of forms adapted to a chosen route of administration, for example, orally, parenterally, intravenously, subcutaneously, and like routes. A preferred method of administration is oral administration.

Fatty Acid Component

The composition of the present invention comprises a fatty acid component. The fatty acid component of the present invention can comprise polyunsaturated fatty acids (PUFAs). PUFAs are fatty acid carbon chains having two or more carbon-carbon double bonds. PUFAs are referred to as essential fatty acids. PUFAs can be divided into omega-3 and omega-6 fatty acids. OMEGA-3 PUFA.

The fatty acid component of the present invention can comprise omega-3 PUFA. Omega-3 fatty acids' first carbon-carbon double bond is at the third carbon from the methyl end. Omega-3 fatty acids are selected from the group comprising alpha-linolenic acid, stearidonic acid, eicosatrienoic acid, eicosatetraenoic acid, eicosapentaenoic acid (EPA), octadecatetraenoic acid, docosapentaenoic acid, docosahexaenoic acid (DHA), and mixtures thereof. The omega-3 fatty acid may exist as a free fatty acid or in a derivatized form such as a monoacylglycerol, diacylglycerol, triacylglycerol, or phospholipids.

When omega-3 is present in the fatty acid component, omega-3 comprises from about from about 0.04% to about 100% by weight of the composition, from about 0.05% to about 95% by weight of the composition, from about 0.1% to about 90% by weight of the composition, from about 0.1% to about 75% by weight of the composition, from about 0.2% to about 70% by weight of the composition, from about 0.2 to about 20% by weight of the composition, from about 0.2% to about 10% by weight of the composition, from about 0.2% to about 2.0% by weight of the composition, from about 0.5% to about 1.0%, by weight of the composition.

Non-limiting sources of omega-3 PUFA for use in the present invention are fish, such as salmon, lake trout, tuna, halibut, mackerel, anchovies, sardine, and herring; fish meal; shellfish; seafood; shrimp; scallops; fish oil such as menhaden oil, cod liver oil, herring oil, mackerel oil, sardine oil, and salmon oil; tofu; algae; eggs; organ meats; squash; chicken; broccoli; wheat germ; pulses, such as peas, beans, and lentils; vegetable oils, such as rapeseed oil, corn oil, safflower oil, sunflower oil, canola oil, flaxseed oil, wheat germ oil, and linseed oil; seeds, such as pumpkin seeds, linseeds, flaxseed and hemp seeds; soybeans; chia (chia sage); perilla (shiso); purslane (portulaca); lingonberry (cowberry); microalgae; brown algae; acai palm fruit; seabuchthorn; leafy vegetables; and nuts, such as walnuts.

DHA

The fatty acid component comprises DHA. DHA is a twenty-two carbon chain omega-3 PUFA containing six carbon-carbon double bonds. The first carbon-carbon double bond is located at the third carbon from the methyl end. DHA can be referred to as 22:6n-3.

The fatty acid component comprises from about 0.04% to about 100% of DHA, by weight of the composition. The fatty acid component comprises DHA from about 0.02% to about 95%, from about 0.1% to about 80%, from about 0.2% to about 75%, from about 0.2% to about 60%, from about 0.2% to about 50%, from about 1.0% to about 40%, from about 1.0% to about 20%, from about 1.0% to about 10% by weight of the composition, from about 2.0% to about 5%, by weight of the composition.

Non-limiting sources of DHA for use in the present invention are fish, such as salmon, lake trout, tuna, halibut, mackerel, anchovies, sardine, and herring; fish meal; shellfish; seafood; shrimp; scallops; fish oil such as menhaden oil, cod liver oil, herring oil, mackerel oil, sardine oil, and salmon oil; tofu; algae; eggs; organ meats; squash; chicken; broccoli; wheat germ; pulses, such as peas, beans, and lentils; vegetable oils, such as rapeseed oil, corn oil, safflower oil, sunflower oil, canola oil, flaxseed oil, wheat germ oil, and linseed oil; seeds, such as pumpkin seeds, linseeds, flaxseed and hemp seeds; soybeans; chia (chia sage); perilla (shiso); purslane (portulaca); lingonberry (cowberry); microalgae; brown algae; acai palm fruit; seabuchthorn; leafy vegetables; and nuts, such as walnuts.

Without being bound by theory, it is believed that the administration to a companion animal of a composition comprising a fatty acid component comprising DHA is effective at improving and treating bone health and muscle health. Bone mineral density, bone mineral content, bone ash content, and osteopenia are indicators of bone health. Administration of said DHA to a companion animal will result in the prevention and/or treatment of osteopenia and/or prevention and/or treatment of the loss of bone ash content and/or prevention and/or treatment of the loss of bone mineral density and/or prevention and/or treatment of the loss of bone mineral content. Muscle fiber diameter, muscle wet weight, muscle mass, and muscle atrophy are indicators of muscle health. Administration of DHA to a companion animal will result in the prevention and/or treatment of muscle atrophy and/or prevention and/or treatment of the loss of muscle fiber diameter and/or prevention and/or treatment of the loss of muscle wet weight and/or prevention and/or treatment of the loss of muscle mass.

EPA

The fatty acid component further comprises EPA. EPA is a twenty carbon chain omega-3 PUFA containing five carbon-carbon double bonds. The first carbon-carbon double bound is located at the third carbon from the methyl end. EPA can be referred to as 20:5n-3.

The fatty acid component comprises from about 0.02% to about 40% of EPA, by weight of the composition. The fatty acid component can comprise EPA from about 0.03% to about 35%, from about 0.1% to about 25%, from about 1.0% to about 10%, from about 2.0% to about 5%, by weight of the composition.

Non-limiting sources of EPA for use in the present invention are fish, such as salmon, lake trout, tuna, halibut, mackerel, anchovies, sardine, and herring; fish meal; shellfish; seafood; shrimp; scallops; fish oil such as menhaden oil, cod liver oil, herring oil, mackerel oil, sardine oil, and salmon oil; tofu; algae; eggs; organ meats; squash; chicken; broccoli; wheat germ; pulses, such as peas, beans, and lentils; vegetable oils, such as rapeseed oil, corn oil, safflower oil, sunflower oil, canola oil, flaxseed oil, wheat germ oil, and linseed oil; seeds, such as pumpkin seeds, linseeds, flaxseed and hemp seeds; soybeans; chia (chia sage); perilla (shiso); purslane (portulaca); lingonberry (cowberry); microalgae; brown algae; acai palm fruit; seabuchthom; leafy vegetables; and nuts, such as walnuts.

The fatty acid component comprises a ratio of DHA to EPA of from about 3:1 to about 25:1. The ratio of DHA to EPA is from about 3:1 to about 22:1, from about 4:1 to about 21:1, from about 5:1 to about 20:1, from about 6:1 to about 18:1, from about 7:1 to about 16:1, from about 8:1 to about 15:1, from about 9:1 to about 14:1, from about 10:1 to about 13:1, from about 11:1 to about 12:1.

Omega-6 PUFA

The fatty acid component of the present invention can comprise omega-6 PUFA. Omega-6 fatty acids' first carbon-carbon double bond is at the sixth carbon from the methyl end. Omega-6 fatty acids are selected from the group comprising linoleic acid (18:2n-6), gamma-linolenic acid (18:3n-6), dihomo-gamma-linolenic acid (20:3n-6), arachidonic acid (20:4n-6), and mixtures thereof. The omega-6 fatty acid may exist as a free fatty acid or in as derivatized form such as a monoacylglycerol, diacylglycerol, triacylglycerol or phospholipid.

When omega-6 is present in the fatty acid component, the composition comprises a ratio of omega-6 to omega-3 of from about 1:0.1 to about 20:1, from about 3:0.5 to about 15:1, from about 3.5:1 to about 12.5:1, from about 5:1 to about 10:1. When omega-6 is present in the fatty acid component, the composition comprises omega-6 from about 0.05% to about 25% by weight of the composition, from about 0.1% to about 20%, from about 0.2% to about 15% by weight of the composition, from about 1.0% to about 10% from about 2.0% to about 5%, by weight of the composition.

Non-limiting sources of omega-6 PUFA for use in the present invention are fish, such as salmon, lake trout, tuna, halibut, and herring; fish meal; shellfish; shrimp; scallops; tofu; squash; broccoli; pulses, such as peas, beans, and lentils; tahini; vegetable oils, such as rapeseed oil, corn oil, safflower oil, sesame oil, hemp oil, pumpkin oil, soybean oil, walnut oil, wheat germ oil, grape seed oil, evening primrose oil, sunflower oil, canola oil, and linseed oil; seeds, such as pumpkin seeds, linseeds, sunflower seeds, safflower seeds, sesame seeds, flaxseed and hemp seeds; soybeans; leafy vegetables; and nuts, such as walnuts and peanuts.

Compositions

It is believed that the fatty acid component comprising DHA and EPA described in the present invention can be added to any composition adapted for administration to a companion animal.

Typical formulae for compositions are well known in the art. In addition to proteinaceous and farinaceous materials, the compositions of the invention generally may include vitamins, minerals, and other additives such as flavorings, preservatives, emulsifiers and humectants. The nutritional balance, including the relative proportions of vitamins, minerals, protein, fat and carbohydrate, is determined according to dietary standards known in the veterinary and nutritional art.

Nonlimiting examples of dry compositions may optionally contain on a dry matter basis, from about 1% to about 50% crude protein, from about 0.5% to about 25% crude fat, from about 1% to about 10% supplemental fiber, all by weight of the composition. The dry composition may have a total moisture content from about 1% to about 30% moisture. Alternatively, a dry composition may contain on a dry matter basis, from about 5% to about 35% crude protein, from about 5% to about 25% crude fat, from about 2% to about 8% supplemental fiber, all by weight of the composition. The dry composition may have a total moisture content from about 2% to about 20% moisture. Alternatively, the dry composition contains on a dry matter basis, a minimum protein level of about from about 9.5% to about 22%, a minimum fat level of from about 8% to about 13%, a minimum supplemental fiber level of from about 3% to about 7%, all by weight of the composition. The dry animal composition may also have a minimum metabolizable energy level of about 3.5 Kcal/g. The dry composition may have a total moisture content from about 3% to about 8%,

Nonlimiting examples of a semi-moist composition may optionally contain on a dry matter basis, from about 0.5% to about 50% crude protein, from about 0.5% to about 25% crude fat, from about 0.5% to about 15% supplemental fiber, all by weight of the composition. The semi-moist composition may have a total moisture content from about 30% to about 50% moisture. Alternatively, the semi-moist compositions may contain on a dry matter basis, from about 5% to about 35% crude protein, from about 5% to about 25% crude fat, from about 1% to about 5% supplemental fiber, and all by weight of the composition. The semi-moist composition may have a total moisture content from about 35% to about 45% moisture. Alternatively, the semi-moist composition may have on a dry mater basis, a minimum protein level of about from about 9.5% to about 22%, a minimum fat level of from about 8% to about 13%, a minimum supplemental fiber level of from about 2% to about 3%, all by weight of the composition. The semi-moist composition may have a total moisture content from about 38% to about 42%. The semi-moist composition may also have a minimum metabolizable energy level of about 3.5 Kcal/g and from about 0.1% to about 20% ash, and from about 0.001% to about 5.0% taurine.

Nonlimiting examples of a moist composition may optionally contain on a dry matter basis, from about 0.5% to about 50% crude protein, from about 0.5% to about 25% crude fat, from about 0.01% to about 15% supplemental fiber, all by weight of the composition. The moist composition may have a total moisture content from about 50% to about 90% moisture. Alternatively, the moist compositions may contain on a dry matter basis, from about 5% to about 35% crude protein, from about 5% to about 25% crude fat, from about 0.05% to about 5% supplemental fiber, all by weight of the composition. The moist composition may have a total moisture content from about 60% to about 85% moisture. Alternatively, a moist animal composition may contain on a dry matter basis, a minimum protein level of about from about 9.5% to about 22%, a minimum fat level of from about 8% to about 13%, a minimum supplemental fiber level of from about 0.1% to about 3%, all by weight of the composition. The moist composition may have a total moisture content from about 65% to about 80%. The moist composition may also have a minimum metabolizable energy level of about 1.0 Kcal/g and from about 0.1% to about 20% ash, and from about 0.001% to about 5.0% taurine.

In one embodiment of the present invention, the composition is a composition, whether dry, moist, semi-moist or otherwise, that comprises on a dry matter basis, from about 5% to about 50%, alternatively 20% to about 50% of animal-derived ingredients, by weight of the composition. Non-limiting examples of animal-derived ingredients include chicken, beef, pork, lamb, turkey (or other animal) protein or fat, egg, fishmeal, and the like.

Where the composition is in the form of a gravy, the composition may comprise at least 10% of a broth, or stock, non-limiting examples of which include vegetable beef, chicken or ham stock. Typical gravy compositions may comprise on a dry matter basis, from about 0.5% to about 5% crude protein, and from about 2% to about 5% crude fat.

Where the composition is in the form of a supplement composition such as biscuits, chews, and other treats, the supplement may comprise, on a dry matter basis, from about 20% to about 60% protein, from about 22% to about 40% protein, by weight of the supplement composition. As another example, the supplement compositions may comprise, on a dry matter basis, from about 5% to about 35% fat, or from about 10% to about 30% fat, by weight of the supplement composition. Compositions and supplement compositions intended for use by animals such as cats or dogs are commonly known in the art.

Optional Ingredients

The composition of the present invention can further comprise a wide range of other optional ingredients.

Non-limiting examples of additional components include animal protein, plant protein, farinaceous matter, vegetables, fruit, egg-based materials, undenatured proteins, food grade polymeric adhesives, gels, polyols, starches, gums, flavorants, seasonings, salts, colorants, time-release compounds, minerals, vitamins, antioxidants, prebiotics, probiotics, aroma modifiers, textured wheat protein, textured soy protein, textured lupin protein, textured vegetable protein, breading, comminuted meat, flour, comminuted pasta, water, and combinations thereof.

Non-limiting examples of optional ingredients can include at least one vegetable. Non-limiting examples of vegetables include carrots, peas, potatoes, cabbage, celery, beans, corn, tomatoes, broccoli, cauliflower, leeks and combinations thereof.

Also useful herein, as an optional ingredient, is a filler. The filler can be a solid, a liquid or packed air. The filler can be reversible (for example thermo-reversible including gelatin) and/or irreversible (for example thermo-irreversible including egg white). Non-limiting examples of the filler include gravy, gel, jelly, aspic, sauce, water, air (for example including nitrogen, carbon dioxide, and atmospheric air), broth, and combinations thereof.

Non-limiting examples of colorants include, but are not limited to, synthetic or natural colorants, and any combination thereof. When present the colorants are from about 0.0001% to about 5%, from about 0.001% to about 1%, from about 0.005% to about 0.1%, on a dry matter basis, of said colorant.

Additionally, probiotic microorganisms, such as Lactobacillus or Bifidobacterium species, for example, may be added to the composition or the compositions themselves.

Also useful herein, as an optional ingredient, is at least one fruit. Non-limiting examples include tomatoes, apples, avocado, pears, peaches, cherries, apricots, plums, grapes, oranges, grapefruit, lemons, limes, cranberries, raspberries, blueberries, watermelon, cantelope, mushmellon, honeydew melon, strawberries, banana, and combinations thereof.

The compositions of the present invention may further comprise a source of carbohydrate. Grains or cereals such as rice, corn, milo, sorghum, barley, wheat, and the like are illustrative sources.

The compositions may also contain other materials such as dried whey and other dairy by products.

Method Of Manufacture

The composition of the present invention may be prepared by any known or otherwise effective technique, suitable for making and formulating the desired composition.

The composition can be processed by a variety of well-known means including steam tunnel, extrusion, freeze-texturization, baking, gelling, retort, microwave heating, ohmic heating, and combinations thereof.

Total Moisture Content Method

The method involves the analysis of the total moisture content in the composition. The analysis is based on the procedure outlined in AOAC method 930.15 and AACC method 44-19.

A composition sample is prepared by taking one unit volume, for example, 375 gram of the composition, and homogenizing in a food processor to a uniform consistency like a paste. A composition larger than 375 gram would be subdivided to create equal and representative fractions of the whole such that a 375 gram sample is obtained.

The paste of the composition is individually sampled in triplicate at a volume less than or equal to 100 ml and placed individually sealed in a 100 ml Nasco Whirl-Pak® (Fort Atkinson, WI 53538-0901). During the process of sealing the Whirl-Pak®, excess air is evacuated manually from the container just prior to final closure thereby minimizing the container headspace. The Whirl-Pak® is closed per manufacturer's instructions—tightly folding the bag over three (3) times and bending the tabs over 180 degrees.

All samples are refrigerated at 6° C. for less than 48 h prior to moisture analysis.

For total moisture analysis, the tare weight of each moisture tin and lid are recorded to 0.0001 g. Moisture tins and lids are handled using dry and clean forceps. Moisture tins and lids are held dry over desiccant in a sealed desiccator. A Whirl-Pak® containing a sample is unfolded and a 2.0000+/−0.2000 gram sample is weighed into the uncovered moisture tin. The weight of the sample in the moisture tin is recorded. The lid is placed atop the moisture tin in an open position to allow moisture loss but contain all other material during air oven drying. The lid and moisture tin loaded with sample are placed in an air oven operating at 135° C. for 6 h. Time is tracked using a count-down timer.

After drying, the tin is removed from the oven and the dried lid is placed atop the tin using forceps. The covered moisture tin with dried sample is placed immediately in a desiccator to cool. The sealed desiccator is filled below the stage with active desiccant. Once cool to room temperature, the covered moisture tin with dried sample is weighed to 0.0001 g and weight recorded. The total moisture content of each sample is calculated using the following formula: Total Moisture Content (%)=100−(weight of tin, lid and sample after drying−empty tin and lid weight)×100/initial sample weight.

### EXAMPLES

The following examples further describe and demonstrate embodiments within the scope of the invention. The examples are given solely for the purpose of illustration and are not to be construed as limitations of the present invention, as many variations thereof are possible without departing from the spirit and scope of the invention. All of the following examples are compositions that comprise a fatty acid component that are utilized by a companion animal.

### Examples 1-30

Dry Compositions (% Dry Matter by Weight of Composition)

| | Ex. 1 | Ex. 2 | Ex. 3 | Ex. 4 | Ex. 5 | Ex. 6 | Ex. 7 | Ex. 8 |
|---|---|---|---|---|---|---|---|---|
| Protein | 32 | 32 | 33 | 31.5 | 20 | 39 | 39 | 41 |
| Carbohydrate | 37.2 | 37.2 | 37.2 | 25 | 5 | 25.3 | 25.3 | 25.59 |
| Fat | 15.35 | 13.36 | 15.27 | 12.72 | 3 | 20.37 | 24.17 | 10.41 |
| DHA | 0.27 | 2.1 | 3.5 | 15 | 55 | 0.25 | 3.4 | 5.7 |
| EPA | 0.08 | 0.24 | 0.31 | 0.68 | 3.1 | 0.08 | 0.43 | 0.6 |
| Omega-6 | 4.3 | 4.3 | 0 | 4.3 | 3.1 | 4.3 | 0 | 6.8 |
| Fiber | 2.7 | 2.7 | 2.7 | 2.7 | 2.7 | 1.6 | 1.6 | 1.6 |
| Minerals | 6.5 | 6.5 | 6.42 | 6.5 | 6.5 | 7.5 | 7.5 | 6.7 |
| Vitamins | 1.6 | 1.6 | 1.6 | 1.6 | 1.6 | 1.6 | 1.6 | 1.6 |
| DHA:EPA | 4:1 | 9:1 | 11:1 | 22:1 | 18:1 | 3:1 | 8:1 | 10:1 |
| ratio | | | | | | | | |

| | Ex. 9 | Ex. 10 | Ex. 11 | Ex. 12 | Ex. 13 | Ex. 14 | Ex. 15 | Ex. 16 |
|---|---|---|---|---|---|---|---|---|
| Protein | 40 | 26 | 28 | 28 | 28 | 28 | 20 | 37 |
| Carbohydrate | 21.7 | 14.7 | 45.93 | 46 | 46 | 44 | 8.2 | 29.7 |
| Fat | 2.5 | 1.7 | 13.79 | 16.21 | 9.5 | 0.3 | 8 | 18.33 |
| DHA | 20 | 45 | 0.21 | 2.1 | 4.5 | 15 | 50 | 0.29 |
| EPA | 0.8 | 1.9 | 0.07 | 0.19 | 0.31 | 0.6 | 2.1 | 0.08 |
| Omega-6 | 4.3 | 0 | 4 | 0 | 3.69 | 4.1 | 3.7 | 4.3 |
| Fiber | 1.6 | 1.6 | 2 | 1.5 | 2 | 2 | 2 | 2.2 |
| Minerals | 7.5 | 7.5 | 5 | 5 | 5 | 5 | 5 | 6.5 |
| Vitamins | 1.6 | 1.6 | 1 | 1 | 1 | 1 | 1 | 1.6 |
| DHA:EPA | 25:1 | 24:1 | 3:1 | 11:1 | 15:1 | 25:1 | 24:1 | 3.6:1 |
| ratio | | | | | | | | |

| | Ex. 17 | Ex. 18 | Ex. 19 | Ex. 20 | Ex. 21 | Ex. 22 | Ex. 23 | Ex. 24 |
|---|---|---|---|---|---|---|---|---|
| Protein | 37 | 37 | 35 | 27 | 29 | 29 | 29 | 39 |
| Carbohydrate | 29.7 | 28 | 15 | 5 | 48.2 | 48 | 47 | 15 |
| Fat | 15.5 | 12.38 | 8.3 | 0.3 | 9.91 | 9 | 7.5 | 4.9 |
| DHA | 3.1 | 8.5 | 28 | 55 | 0.3 | 1.74 | 4.1 | 30 |
| EPA | 0.34 | 0.62 | 1.4 | 2.4 | 0.09 | 0.2 | 0.28 | 1.3 |
| Omega-6 | 4.3 | 3.2 | 2 | 0 | 2.7 | 2.96 | 2.7 | 0 |
| Fiber | 2.2 | 2.2 | 2.2 | 2.2 | 2.2 | 2 | 2.2 | 2.2 |
| Minerals | 6.26 | 6.5 | 6.5 | 6.5 | 6.5 | 6 | 6.12 | 6.5 |
| Vitamins | 1.6 | 1.6 | 1.6 | 1.6 | 1.1 | 1.1 | 1.1 | 1.1 |
| DHA:EPA | 9:1 | 14:1 | 20:1 | 23:1 | 3:1 | 9:1 | 15:1 | 23:1 |
| ratio | | | | | | | | |

| | Ex. 25 | Ex. 26 | Ex. 27 | Ex. 28 | Ex. 29 | Ex. 30 |
|---|---|---|---|---|---|---|
| Protein | 12 | 39 | 39 | 39 | 40 | 21 |
| Carbohydrate | 8 | 32.2 | 32.24 | 32.2 | 14 | 8.4 |
| Fat | 2.2 | 16 | 15.4 | 14.57 | 9 | 2.1 |
| DHA | 60 | 0.31 | 0.87 | 1.74 | 24 | 56 |
| EPA | 2.8 | 0.09 | 0.09 | 0.09 | 0.97 | 2.5 |
| Omega-6 | 5.2 | 1.6 | 1.6 | 1.6 | 1.23 | 0 |
| Fiber | 2.2 | 2.7 | 2.7 | 2.7 | 2.7 | 2.4 |
| Minerals | 6.5 | 6.5 | 6.5 | 6.5 | 6.5 | 6.2 |
| Vitamins | 1.1 | 1.6 | 1.6 | 1.6 | 1.6 | 1.4 |
| DHA:EPA | 21:1 | 3:1 | 10:1 | 19:1 | 25:1 | 22:1 |
| ratio | | | | | | |

The dry compositions of Examples 1-5 are puppy compositions. The dry compositions of Examples 6-10 are kitten compositions. The dry compositions of Examples 11-15 are adult dog compositions. The dry compositions of Examples 16-20 are adult cat compositions. The dry compositions of Examples 21-25 are senior dog compositions. The dry compositions of Examples 26-30 are senior cat compositions.

The dry compositions of Examples 1-10 can be made by first, milling and mixing the carbohydrate sources with the vitamins, minerals, and fiber sources. Then, add the mixture to the protein sources. Extrude the ingredients into kibbles. Dry the kibbles. Place the kibbles in a tumble drum and tumble while spraying the DHA and EPA source(s) into the drum. Package the finished product.

The dry compositions of Examples 11-20 can be made by first, mill and mix the carbohydrate sources with the vitamins, minerals, and fiber sources. Add the DHA and EPA source(s) to the mixture. Then, add the mixture to the protein sources. Extrude the ingredients into kibbles. Dry the kibbles. Package the finished product.

The dry compositions of Examples 21-30 can be made by first, milling and mixing the carbohydrate sources with the vitamins, minerals, and fiber sources. Add the DHA and EPA source(s) to the mixture. Then, add the mixture to the protein sources. Extrude the ingredients into kibbles. Dry the kibbles. Place the kibbles in a tumble drum and tumble while spraying the DHA and EPA source(s) into the drum. Package the finished product.

### Examples 31-60

Wet Compositions (% Dry Matter by Weight of Composition)

| | Ex. 31 | Ex. 32 | Ex. 33 | Ex. 34 | Ex. 35 | Ex. 36 | Ex. 37 | Ex. 38 |
|---|---|---|---|---|---|---|---|---|
| Protein | 47 | 47 | 47 | 40 | 35 | 48 | 48 | 48 |
| Carbohydrate | 12.5 | 15.5 | 10.5 | 12 | 8.8 | 10 | 10 | 10 |
| Fat | 23.95 | 23.63 | 20.3 | 13.53 | 2 | 28.16 | 25.63 | 25.5 |
| DHA | 0.19 | 1.25 | 5.5 | 18 | 40 | 0.18 | 3.5 | 6.1 |
| EPA | 0.06 | 0.12 | 0.4 | 0.97 | 1.7 | 0.06 | 0.28 | 0.4 |
| Omega-6 | 3.8 | 0 | 3.8 | 3 | 0 | 3.6 | 3.6 | 0 |
| Fiber | 3.1 | 3.1 | 3.1 | 3.1 | 3.1 | 0.9 | 0.9 | 0.9 |
| Minerals | 7.8 | 7.8 | 7.8 | 7.8 | 7.8 | 7.6 | 6.59 | 7.6 |
| Vitamins | 1.6 | 1.6 | 1.6 | 1.6 | 1.6 | 1.5 | 1.5 | 1.5 |
| DHA:EPA | 3:1 | 10:1 | 14:1 | 19:1 | 24:1 | 3:1 | 13:1 | 15:1 |
| ratio | | | | | | | | |
| | Ex. 39 | Ex. 40 | Ex. 41 | Ex. 42 | Ex. 43 | Ex. 44 | Ex. 45 | Ex. 46 |
| Protein | 45 | 25 | 39 | 43 | 39 | 40 | 26 | 46 |
| Carbohydrate | 14.7 | 7.5 | 23.95 | 22 | 23.5 | 17 | 8 | 13.8 |
| Fat | 5 | 1 | 22.72 | 23.57 | 18.44 | 7.9 | 0 | 26.23 |
| DHA | 21 | 54 | 0.25 | 1.32 | 4.8 | 20 | 50 | 0.29 |
| EPA | 1.2 | 2.5 | 0.08 | 0.11 | 0.3 | 1.1 | 2 | 0.08 |
| Omega-6 | 3.1 | 0 | 4 | 0 | 4 | 4 | 4 | 4.4 |
| Fiber | 0.9 | 0.9 | 2 | 2 | 2 | 2 | 4 | 1.2 |
| Minerals | 7.6 | 7.6 | 6 | 6 | 5.96 | 6 | 4 | 6 |
| Vitamins | 1.5 | 1.5 | 2 | 2 | 2 | 2 | 2 | 2 |
| DHA:EPA | 18:1 | 22:1 | 3:1 | 12:1 | 16:1 | 18:1 | 25:1 | 4:1 |
| ratio | | | | | | | | |
| | Ex. 47 | Ex. 48 | Ex. 49 | Ex. 50 | Ex. 51 | Ex. 52 | Ex. 53 | Ex. 54 |
| Protein | 46 | 46 | 41 | 25 | 42 | 42 | 42 | 35 |
| Carbohydrate | 13.8 | 13.8 | 13.65 | 11 | 30.5 | 30.02 | 30.25 | 20 |
| Fat | 25.72 | 29.32 | 5 | 2 | 14.65 | 14.09 | 10 | 7 |
| DHA | 0.8 | 1.6 | 26 | 48 | 0.24 | 3.1 | 4.8 | 24 |
| EPA | 0.08 | 0.08 | 1.15 | 2 | 0.08 | 0.29 | 0.35 | 1.4 |
| Omega-6 | 4.4 | 0 | 4 | 2.8 | 2.1 | 0 | 2.1 | 2.1 |
| Fiber | 1.2 | 1.2 | 1.2 | 1.2 | 1.3 | 1.3 | 1.3 | 1.3 |
| Minerals | 6 | 6 | 6 | 6 | 6.63 | 6.7 | 6.7 | 6.7 |
| Vitamins | 2 | 2 | 2 | 2 | 2.5 | 2.5 | 2.5 | 2.5 |
| DHA:EPA | 10:1 | 20:1 | 23:1 | 24:1 | 3:1 | 11:1 | 14:1 | 17:1 |
| ratio | | | | | | | | |

| | Ex. 55 | Ex. 56 | Ex. 57 | Ex. 58 | Ex. 59 | Ex. 60 |
|---|---|---|---|---|---|---|
| Protein | 20 | 47.91 | 48 | 47.24 | 40 | 16 |
| Carbohydrate | 10 | 12.5 | 12.5 | 12.5 | 20 | 12.1 |
| Fat | 1.9 | 27.05 | 25.04 | 23.5 | 4.3 | 0 |
| DHA | 51 | 0.26 | 2.1 | 5.9 | 22 | 57 |
| EPA | 2.4 | 0.08 | 0.16 | 0.36 | 1.2 | 2.3 |
| Omega-6 | 4.2 | 1.7 | 1.7 | 0 | 2 | 2.1 |
| Fiber | 1.3 | 1.3 | 1.3 | 1.3 | 1.3 | 1.3 |
| Minerals | 6.7 | 7.1 | 7.1 | 7.1 | 7.1 | 7.1 |
| Vitamins | 2.5 | 2.1 | 2.1 | 2.1 | 2.1 | 2.1 |
| DHA:EPA | 21:1 | 3:1 | 13:1 | 16:1 | 18:1 | 25:1 |
| ratio | | | | | | |

The wet compositions of Examples 31-35 are puppy compositions. The wet compositions of Examples 36-40 are kitten compositions. The wet compositions of Examples 41-45 are adult dog compositions. The wet compositions of Examples 46-50 are adult cat compositions. The wet compositions of Examples 51-55 are senior dog compositions. The wet compositions of Examples 56-60 are senior cat compositions.

The wet compositions of Examples 31-60 can be made by first drying and milling the carbohydrate sources. Mix carbohydrate sources, vitamins, minerals and fiber sources. Add the DHA and EPA source(s) to the mixture. Blend the mixture with the protein sources. The mixture is packaged into cans and cooked via retort process to provided finished product. For preformed pieces (chunks in gravy) mixture is extruded, passed through a steam tunnel for preconditioning, cut to desired shape, packaged with added water and retorted to provide safe finished product.

### Examples 61-90

Moist Compositions (% Dry Matter by Weight of Composition)

| | Ex. 61 | Ex. 62 | Ex. 63 | Ex. 64 | Ex. 65 | Ex. 66 | Ex. 67 | Ex. 68 |
|---|---|---|---|---|---|---|---|---|
| Protein | 31 | 31 | 31 | 30 | 25 | 35 | 36 | 32 |
| Carbohydrate | 37.3 | 34.8 | 35.4 | 35.6 | 11 | 25.7 | 25.7 | 25.7 |
| Fat | 16.41 | 18.5 | 12.3 | 3 | 2 | 20.4 | 17.5 | 19.6 |
| DHA | 0.22 | 2.7 | 4.2 | 15 | 42 | 0.23 | 3.1 | 5.9 |
| EPA | 0.07 | 0.33 | 0.32 | 0.8 | 1.7 | 0.07 | 0.51 | 0.31 |
| Omega-6 | 4.3 | 0 | 4.3 | 4.9 | 5.8 | 4.3 | 4.3 | 0 |
| Fiber | 2.4 | 2.9 | 2.9 | 2.9 | 2.9 | 1.6 | 1.6 | 1.6 |
| Minerals | 6 | 6.4 | 6.4 | 5.4 | 6.4 | 7.1 | 7.1 | 7.1 |
| Vitamins | 1.3 | 1.4 | 1.4 | 1.4 | 1.4 | 1.6 | 1.6 | 1.6 |
| Humectants | 1 | 1.97 | 1.78 | 1 | 1.8 | 4 | 2.59 | 6.19 |
| DHA:EPA | 3:1 | 8:1 | 13:1 | 19:1 | 25:1 | 3:1 | 6:1 | 19:1 |
| ratio | | | | | | | | |
| | Ex. 69 | Ex. 70 | Ex. 71 | Ex. 72 | Ex. 73 | Ex. 74 | Ex. 75 | Ex. 76 |
| Protein | 30 | 18.8 | 28 | 28 | 27 | 20 | 20 | 37 |
| Carbohydrate | 16 | 9 | 45 | 44 | 44 | 35 | 7 | 30 |
| Fat | 5.3 | 0 | 13.72 | 12.5 | 14.3 | 5 | 0 | 18.39 |
| DHA | 28 | 53 | 0.21 | 1.43 | 2.6 | 22 | 61 | 0.24 |
| EPA | 1.3 | 2.2 | 0.07 | 0.15 | 0.16 | 0.97 | 2.51 | 0.07 |
| Omega-6 | 5.1 | 6.7 | 4 | 4 | 0 | 4 | 0 | 4.3 |
| Fiber | 1.6 | 1.4 | 2 | 2 | 2 | 1.03 | 2 | 2.1 |
| Minerals | 7.1 | 6.8 | 5 | 5 | 5 | 5 | 5 | 6.2 |
| Vitamins | 1.6 | 1.2 | 1 | 1 | 1 | 1 | 1 | 1.1 |
| Humectants | 4 | 0.9 | 1 | 1.92 | 3.94 | 6 | 1.49 | 0.6 |
| DHA:EPA | 22:1 | 24:1 | 3:1 | 10:1 | 16:1 | 23:1 | 24:1 | 3:1 |
| ratio | | | | | | | | |
| | Ex. 77 | Ex. 78 | Ex. 79 | Ex. 80 | Ex. 81 | Ex. 82 | Ex. 83 | Ex. 84 |
| Protein | 37 | 37 | 36 | 14 | 29 | 29 | 29 | 26 |
| Carbohydrate | 30.4 | 28 | 25 | 10 | 46.1 | 40 | 45 | 30 |
| Fat | 20.4 | 14.5 | 3 | 2 | 9.94 | 8 | 9.06 | 2 |
| DHA | 1.8 | 4.6 | 19 | 57 | 0.27 | 2.1 | 3.7 | 25.4 |
| EPA | 0.2 | 0.37 | 1.11 | 2.3 | 0.09 | 0.2 | 0.24 | 1.4 |
| Omega-6 | 0 | 4.3 | 4.3 | 3.3 | 2.7 | 2.7 | 0 | 3.1 |
| Fiber | 2.1 | 2.1 | 2.1 | 2.1 | 2.1 | 2.1 | 2.1 | 2.1 |
| Minerals | 6.4 | 6.4 | 6.4 | 6.4 | 6.4 | 6.4 | 6.4 | 6.4 |
| Vitamins | 1.1 | 1.1 | 1.1 | 1.1 | 1.4 | 1.4 | 1.4 | 1.4 |
| Humectants | 0.6 | 1.63 | 1.99 | 1.8 | 2 | 8.1 | 3.1 | 2.2 |
| DHA:EPA | 9:1 | 12:1 | 17:1 | 25:1 | 3:1 | 11:1 | 15:1 | 18:1 |
| ratio | | | | | | | | |

| | Ex. 85 | Ex. 86 | Ex. 87 | Ex. 88 | Ex. 89 | Ex. 90 |
|---|---|---|---|---|---|---|
| Protein | 24 | 39 | 39 | 39 | 35 | 25 |
| Carbohydrate | 10 | 30.1 | 29.1 | 28 | 21 | 10 |
| Fat | 3 | 17.03 | 17.5 | 15.6 | 3.13 | 1 |
| DHA | 41.6 | 0.28 | 1.2 | 3.7 | 20 | 50 |
| EPA | 1.7 | 0.09 | 0.17 | 0.23 | 0.97 | 2 |
| Omega-6 | 4 | 1.6 | 0 | 1.6 | 1 | 0.5 |
| Fiber | 2.1 | 2.9 | 2.9 | 2.5 | 2.9 | 2.9 |
| Minerals | 6.4 | 6.4 | 6.4 | 6.1 | 6.4 | 6.4 |
| Vitamins | 1.4 | 1.6 | 1.6 | 1.4 | 1.6 | 1.6 |
| Humectants | 5.8 | 1 | 2.13 | 1.87 | 8 | 0.6 |
| DHA:EPA | 25:1 | 3:1 | 7:1 | 16:1 | 21:1 | 25:1 |

The moist compositions of Examples 61-65 are puppy compositions. The moist compositions of Examples 66-70 are kitten compositions. The moist compositions of Examples 71-75 are adult dog compositions. The moist compositions of Examples 76-80 are adult cat compositions. The moist compositions of Examples 81-85 are senior dog compositions. The moist compositions of Examples 86-90 are senior cat compositions.

The moist compositions of Examples 61-90 can be made by milling and mixing the carbohydrate sources with vitamins, minerals, and fiber sources. Add the DHA and EPA source(s) to the mixture. Then, add the mixture to the protein sources with humectants to control water activity and mold. Extrude the ingredients into desired shape. Package the finished product.

### Examples 91-95

Treat Compositions (% Dry Matter by Weight of Composition)

| | Ex. 91 | Ex. 92 | Ex. 93 | Ex. 94 | Ex. 95 |
|---|---|---|---|---|---|
| Protein | 30 | 27.64 | 30 | 12 | 11 |
| Carbohydrate | 50.9 | 53.3 | 50.9 | 40 | 25 |
| Fat | 4.57 | 1.94 | 2.71 | 1.4 | 0 |
| DHA | 0.1 | 2.5 | 4 | 30 | 46 |
| EPA | 0.03 | 0.22 | 0.29 | 1.4 | 1.9 |
| Omega-6 | 2.3 | 2.3 | 0 | 3.1 | 4 |
| Fiber | 3.3 | 3.3 | 3.3 | 3.3 | 3.3 |
| Minerals | 6.6 | 6.6 | 6.6 | 6.6 | 6.6 |
| Vitamins | 2.2 | 2.2 | 2.2 | 2.2 | 2.2 |
| DHA:EPA | 3:1 | 11:1 | 14:1 | 21:1 | 24:1 |
| ratio | | | | | |

The treat compositions of Examples 91-95 can be made by first, mill and mix the dry ingredients (can be the carbohydrate, vitamin, mineral, protein, fiber, and/or DHA and EPA source(s)). Add the liquid ingredients (can be the carbohydrate, vitamin, mineral, protein, fiber, and/or DHA and EPA source(s)). Place mixture in mold of desired shape. Bake mixture in mold. Dry molded mixture and release from mold. Package the finished product.

### Examples 96-100

Gravy Compositions (% Dry Matter by Weight of Composition)

| | Ex. 96 | Ex. 97 | Ex. 98 | Ex. 99 | Ex. 100 |
|---|---|---|---|---|---|
| Protein | 32.15 | 33 | 33 | 32 | 20 |
| Carbohydrate | 10.3 | 10.3 | 10.3 | 10 | 9.06 |
| Fat | 28.72 | 26.75 | 30.23 | 3.1 | 1 |
| DHA | 0.7 | 1.8 | 2.6 | 25 | 40 |
| EPA | 0.23 | 0.25 | 0.17 | 1.2 | 1.64 |
| Omega-6 | 4.2 | 4.2 | 0 | 5 | 4.6 |
| Fiber | 0.8 | 0.8 | 0.8 | 0.8 | 0.8 |
| Minerals | 20.8 | 20.8 | 20.8 | 20.8 | 20.8 |
| Vitamins | 2.1 | 2.1 | 2.1 | 2.1 | 2.1 |
| DHA:EPA | 3:1 | 7:1 | 15:1 | 21:1 | 24:1 |
| ratio | | | | | |

The gravy compositions of Examples 96-100 can be made by first, mill and mix the dry ingredients (can be the carbohydrate, vitamin, mineral, protein, fiber, and/or DHA and EPA source(s)). Add the liquid ingredients (can be the carbohydrate, vitamin, mineral, protein, fiber, and/or DHA and EPA source(s)). Thermally sterilize the mixture. Package the finished product.

### Examples 101-102

Pill Compositions (% Dry Matter by Weight of Composition)

| | Ex. | Ex. |
|---|---|---|
| | 101 | 102 |
| Protein | 0 | 0 |
| Carbohydrate | 0 | 0 |
| Fat | 0 | 0 |
| DHA | 71 | 81 |
| EPA | 4 | 4 |
| Omega-6 | 25 | 15 |
| Fiber | 0 | 0 |
| Minerals | 0 | 0 |
| Vitamins | 0 | 0 |
| DHA:EPA | 19:1 | 20:1 |

The pill compositions of Examples 101-102 can be made by first combining the DHA and EPA source(s) with suitable pharmaceutically-accepted excipients. The term “pharmaceutically-acceptable excipients” as used herein includes any physiologically inert, pharmacologically inactive material known to one skilled in the art, which is compatible with the physical and chemical characteristics of the particular active ingredient selected for use. The mixture is formed into granules. Then, coat the granules and compress the granules into a tablet using apparatuses and/or methods which are well-known to those skilled in the art. Next, film-coat the tablets. Any film-coating which is soluble in the gastric contents pH 1.2-5 can be used. Package the finished product.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

All documents cited in the Detailed Description of the Invention are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to the term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method of improving bone health and muscle health in a companion animal comprising: administering a composition comprising a fatty acid component to a companion animal; and wherein said fatty acid component comprises a ratio of docosahexaenoic acid (DHA) to eicosapentaenoic acid (EPA) from about 3:1 to about 25:1.

2. The method of claim 1, wherein said improving bone health comprises preventing or treating the loss of bone mineral density, preventing or treating the loss of bone mineral content, or preventing or treating the loss of bone ash content or preventing or treating osteopenia; and wherein said improving muscle health comprises preventing or treating the loss of muscle fiber diameter, preventing or treating the loss of muscle mass, or preventing or treating the loss of muscle wet weight or preventing or treating muscle atrophy.

3. The method of claim 1, wherein docosahexaenoic acid (DHA) is from about 0.04% to about 100%, by weight of the composition.

4. The method of claim 1, wherein eicosapentaenoic acid (EPA) is from about 0.02% to about 40% by weight of the composition.

5. The method of claim 1, wherein said ratio of docosahexaenoic acid (DHA) to eicosapentaenoic acid (EPA) is from about 3:1 to about 22:1.

6. The method of claim 1, wherein said ratio of docosahexaenoic acid (DHA) to eicosapentaenoic acid (EPA) is from about 4:1 to about 21:1.

7. The method of claim 1, wherein said ratio of said docosahexaenoic acid (DHA) to said eicosapentaenoic acid (EPA) is from about 5:1 to about 20:1.

8. The method of claim 1, wherein said ratio of docosahexaenoic acid (DHA) to eicosapentaenoic acid (EPA) is from about 6:1 to about 18:1.

9. The method of claim 1, wherein said fatty acid component further comprises an omega-6 polyunsaturated fatty acid.

10. The method of claim 1, wherein said composition is selected from the group consisting of wet, moist, dry, and combinations thereof.

11. The method of claim 1, wherein said composition is selected from the group consisting of pet food, dog food, cat food, supplements, treats, chews, beverages, sauces, pills, capsules and combinations thereof.

12. The method of claim 11, wherein said composition is complete and nutritionally balanced.

13. The method of claim 1, wherein said composition further comprises animal protein, plant protein, farinaceous matter, vegetables, fruit, egg-based materials, undenatured proteins, food grade polymeric adhesives, gels, polyols, starches, gums, flavorants, seasonings, salts, colorants, time-release compounds, delayed release compounds, site specific release compounds, minerals, vitamins, antioxidants, prebiotics, probiotics, aroma modifiers, and combinations thereof.

14. A composition that improves bone health and muscle health in a companion animal comprising: a composition comprising a fatty acid component comprising docosahexaenoic acid (DHA) and eicosapentaenoic acid (EPA); and wherein said fatty acid component comprises a ratio of docosahexaenoic acid (DHA) to eicosapentaenoic acid (EPA) from about 3:1 to about 25:1.

15. The composition of claim 14, wherein docosahexaenoic acid (DHA) is from about 0.04% to about 100%, by weight of the composition.

16. The composition of claim 14, wherein eicosapentaenoic acid (EPA) is from about 0.02% to about 40% by weight of the composition.

17. The composition of claim 14, wherein said ratio of docosahexaenoic acid (DHA) to eicosapentaenoic acid (EPA) is from about 3:1 to about 22:1.

18. The composition of claim 14, wherein said ratio of docosahexaenoic acid (DHA) to eicosapentaenoic acid (EPA) is from about 4:1 to about 21:1.

19. The composition of claim 14, wherein said ratio of docosahexaenoic acid (DHA) to eicosapentaenoic acid (EPA) is from about 5:1 to about 20:1.

20. The composition of claim 14, wherein said ratio of docosahexaenoic acid (DHA) to eicosapentaenoic acid (EPA) is from about 6:1 to about 18:1.

21. The composition of claim 14, wherein said fatty acid component further comprises an omega-6 polyunsaturated fatty acid.

22. The composition of claim 14, wherein said composition is selected from the group consisting of wet, moist, dry, and combinations thereof.

23. The composition of claim 14, wherein said composition is selected from the group consisting of pet food, dog food, cat food, supplements, treats, chews, beverages, sauces, pills, capsules, and combinations thereof.

24. The composition of claim 23, wherein said composition is complete and nutritionally balanced.

25. The composition of claim 14, wherein said composition further comprises animal protein, plant protein, farinaceous matter, vegetables, fruit, egg-based materials, undenatured proteins, food grade polymeric adhesives, gels, polyols, starches, gums, flavorants, seasonings, salts, colorants, time-release compounds, delayed release compounds, site specific release compounds, minerals, vitamins, antioxidants, prebiotics, probiotics, aroma modifiers, and combinations thereof.
